(19) United States
(12) Patent Application Publication
(10) Pub. No.: US 2022/0305146 A1
(43) Pub. Date: Sep. 29, 2022

(54) RADIOPHARMACEUTICAL COMPOSITIONS AND METHODS FOR INFECTIOUS DISEASE DIAGNOSIS AND THERAPY

(71) Applicant: Vyripharm Enterprises, Inc., Houston, TX (US)

(72) Inventor: Jerry L. BRYANT, JR., Bellaire, TX (US)

(21) Appl. No.: 17/697,401

(22) Filed: Mar. 17, 2022

Related U.S. Application Data
(60) Provisional application No. 63/166,910, filed on Mar. 26, 2021.

(51) Int. Cl. A61K 51/04

(52) U.S. Cl. CPC A61K 51/0497; A61K 51/0459; A61K 51/0482

(57) ABSTRACT

Theranostic radiopharmaceutical compositions and methods for targeted infectious disease diagnosis and treatment are provided. The theranostic radiopharmaceutical compositions may include a conjugate of a nucleoside analog, a chelator, and a radionuclide label.

### CROSS-REFERENCE TO RELATED APPLICATIONS

This application claims the benefit of priority to U.S. Provisional Application Ser. No. 63/166,910, filed Mar. 26, 2021, the entire contents of which is hereby incorporated by reference, for all purposes, in its entirety.

### FIELD OF TECHNOLOGY

The present disclosure is directed to radiopharmaceutical compositions and methods for targeted infectious disease diagnosis as well as theranostic radiopharmaceutical compositions and methods for the treatment of infectious diseases. The present disclosure is also related to radiopharmaceutical compositions that include a conjugate of a nucleoside analog, a chelator, and a radionuclide label. The presently disclosed radiopharmaceutical compositions and methods may be particularly useful for diagnostic and prognostic applications in infectious diseases as well as for use in the treatment of infectious diseases.

### BACKGROUND

Medical imaging techniques are critical to the proper diagnosis and monitoring of many diseases, including infectious diseases. Nonetheless, the diagnosis and treatment of many infectious diseases continues to be poorly understood. In particular, since the COVID-19 outbreak was declared a public health emergency of international concern by the World Health Organization (WHO) on Jan. 30, 2020, the progression of the severe acute respiratory syndrome coronavirus 2 (SARS-COV-2) virus has reminded us of the critical role of an effective host immune response as well as the devastating effect of immune dysregulation. Accordingly, there is a critical need for an efficient approach for both the diagnosis and treatment of infectious diseases, such as COVID-19. However, inconsistencies in the diagnosis and stage identification of infectious diseases and their progression persist making identification of appropriate and efficient treatment protocols challenging. Furthermore, effective approaches to tailored or targeted treatment of infectious diseases has also been challenging, including attempts at developing vaccines. Accordingly, there is a need for improved approaches for the accurate diagnosis as well as evaluation of disease progression and severity in order to provide patient-specific targeted treatment protocols and methodologies. The development of radiopharmaceutical analogs for use in infectious disease applications has focused on either diagnostic imaging agents or therapeutic agents. There is a need for theranostic radiopharmaceutical analogs that are effective for use in both diagnostic imaging as well as therapy or treatment of infectious diseases.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 is exemplary process for the synthesis of the compound according to structural formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide, according to an exemplary embodiment of the present disclosure; and

FIG. 2 is an exemplary process for the synthesis of the compound according to structural formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide], according to an exemplary embodiment of the present disclosure.

### DETAILED DESCRIPTION

It will be appreciated that numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein.

The present disclosure provides radiopharmaceutical compositions and methods for diagnosing and treating infectious diseases. It has been unexpectedly discovered that the presently disclosed radiopharmaceutical compositions may be simultaneously used to both treat and diagnose infectious diseases. In particular, the presently disclosed theranostic radiopharmaceutical compositions may effectively be used as radionuclide labels in diagnostic applications, particularly medical imaging applications. Additionally, the same theranostic radiopharmaceutical compositions have been found to exhibit antiviral activity against infectious disease causing agents, such as the virus responsible for COVID-19. The theranostic radiopharmaceutical compositions and methods using the compositions are amenable to providing personalized treatment that is critical to the effective treatment of infectious diseases in individuals that may respond differently to the infectious agents and various treatment protocols. The methods and compositions are safe and cost-effective and the diagnostic methods are non-invasive.

The presently disclosed theranostic radiopharmaceutical compositions may include a conjugate of a nucleoside analog, a chelator, and a label. The nucleoside analog may be a guanine analog. In other cases, the nucleoside analog may be a cell replication check point ligand. In some instances, the nucleoside analog may be a synthetic analog. In other instances, the nucleoside analog may be a natural analog. In some cases, the nucleoside analog may be guanine. According to at least one aspect, the nucleoside analog may be selected from the group consisting of adenine, adenosine, deoxyadenosine, guanine, guanosine, deoxyguanosine, thymine, 5-methyluridine, thymidine, uracile, uridine, deoxyuridine, cytosine, cytidine, deoxycytidine, and any combination thereof. The nucleoside analog may, in some instances, be arabinosyl nucleoside.

In some instances, the chelator may be an aminated chelator or an acid chelator. In some instances, the chelator may be a N4 chelator or ligand. The chelator, may be, for example, cyclam, 6-carboxy-1,4,8,11-tetraazaundecane, or 1,4,8,11-tetraazabicyclohexadecane.

The label may be a radionuclide label. The radionuclide label may be selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof. In at least some instances, the radionuclide label may be configured to facilitate contrast-enhanced imaging when administered to a mammalian subject in conjunction with diagnostic imaging.

The conjugate may be a N4-guanine (N4amG) such as cyclam-am-guanine. In some instances, the conjugate may comprise N-(4-(2-amino-6-oxo-1,6,-dihydro-9H-purin-9-yl)-2-(hydroxymethyl)butyl)-2-(1,4,8,11-tetraazacyclotetradecan-1-yl)acetamide, corresponding to a compound characterized by the structure according to Formula I:

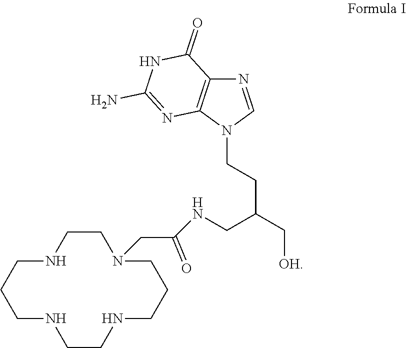 [SMILES: Nc1nc2c(ncn2CCC(CO)CNC(=O)CN2CCCNCCNCCCNCC2)c(=O)[nH]1]

In other instances, the conjugate may comprise N-(9-(4-amino-3-(hydroxymethyl)butyl)-6-oxo-6,9-dihydro-1H-purin-2-yl)-2-(1,4,8,11-tetraazacyclotetradecan-1-yl)acetamide, corresponding to a compound characterized by the structure according to Formula II:

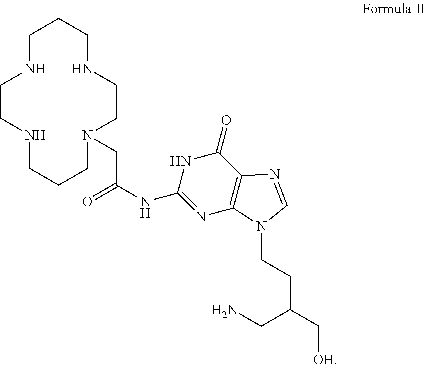 [SMILES: NCC(CO)CCn1cnc2c(=O)[nH]c(NC(=O)CN3CCCNCCNCCCNCC3)nc21]

In still other instances, the conjugate may comprise N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide, corresponding to a compound characterized by the structure according to Formula III:

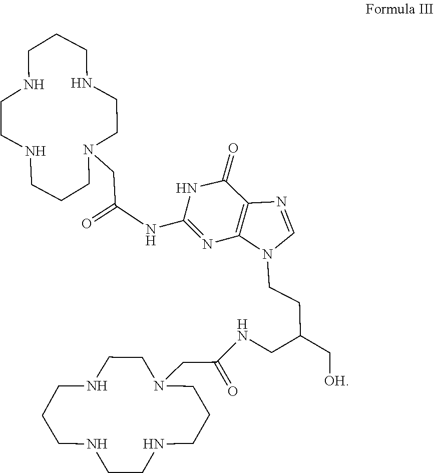 [SMILES: O=C(CN1CCCNCCNCCCNCC1)NCC(CO)CCn1cnc2c(=O)[nH]c(NC(=O)CN3CCCNCCNCCCNCC3)nc21]

In still other instances, the conjugate may comprise 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide], corresponding to a compound characterized by the structure according to Formula IV:

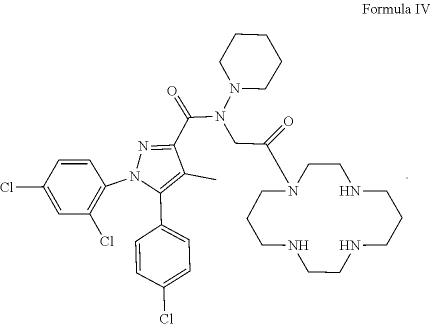 [SMILES: Cc1c(C(=O)N(CC(=O)N2CCCNCCNCCCNCC2)N2CCCCC2)nn(-c2ccc(Cl)cc2Cl)c1-c1ccc(Cl)cc1]

Any of the conjugates disclosed herein, such as the conjugates according to Formula I, Formula II, Formula III, Formula IV, may further be labeled with a radionuclide label to form the theranostic radiopharmaceutical compositions comprising a conjugate of a nucleoside analog, a chelator, and a label. For example, the conjugates in Formulas I-IV may include a label selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

The presently disclosed theranostic radiopharmaceutical compositions may be used in various methods for diagnosing or treating infectious diseases. In particular, the present disclosure provides methods for diagnosing and treating an infectious disease in a subject in need thereof using a theranostic radiopharmaceutical. The method may include administering to the subject a pharmaceutically effective amount of one or more of the presently disclosed theranostic radiopharmaceutical compositions. The method may also include performing an imaging technique on the subject or a portion thereof which is capable of detecting one or more signals from the theranostic radiopharmaceutical composition.

The present disclosure also provides methods for simultaneously monitoring and treating an infectious disease in a subject in need thereof. The methods may include administering to the subject a pharmaceutically effective amount of one or more of the presently disclosed theranostic radiopharmaceutical compositions. The method may also include performing an imaging technique on the subject or a portion thereof which is capable of detecting one or more signals from the theranostic radiopharmaceutical composition.

The present disclosure also provides methods for imaging a plurality of cells in a subject wherein the plurality of cells are infected with an infectious disease pathogen. The methods may include administering to the subject a pharmaceutically effective amount of one or more of the presently disclosed theranostic radiopharmaceutical compositions in a manner such that the plurality of cells effectively receive the composition. The method may also include performing an imaging technique on at least a portion of the subject containing the plurality of cells which is capable of detecting one or more signals from the theranostic radiopharmaceutical composition. The presently disclosed methods may also include making at least one treatment decision based on the results of the imaging technique performed on the subject.

The imaging technique used in any of the presently disclosed methods may be any imaging technique capable of detecting one or more signals from the image probe composition. For example, the imaging technique may be selected from the group consisting of positron emission tomography (PET), computed tomography (CT), single photon emission computed tomography (SPECT), magnetic resonance imaging (MRI), near-infrared (NIR), optical imaging, optoacoustic imaging, ultrasound, and any combination thereof.

In at least some instances, the infectious disease is a viral infection. For instance, the infectious disease may be a respiratory viral infection selected from the group consisting of human influenza, the common cold, Middle East respiratory syndrome (MERS), severe acute respiratory syndrome coronavirus (SARS), and COVID-19. The infectious disease may also be caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus 0C43 (HCoV-0C43), human coronavirus HKU1 (HCoV HKU1), and human coronavirus 229E (HCoV-229E).

As used herein, the term “theranostic,” in all of its forms, refers to a single compound or conjugate that may be used to both treat a condition or disease (e.g., exhibits antiviral activity) and as a radiopharmaceutical capable of facilitating diagnostic imaging of the same condition or disease. As used herein, the term “conjugate,” in all its forms, refers to a compound formed by the joining of two or more chemical compounds. The term “pharmaceutically acceptable derivative,” as used herein, refers to and includes any pharmaceutically acceptable salt, pro-drug, metabolite, ester, ether, hydrate, polymorph, solvate, complex, and adduct of a compound described herein, which, upon administration to a subject, is capable of providing (directly or indirectly) the active ingredient. For example, the term “a pharmaceutically acceptable derivative” of compounds described herein includes all derivatives of the compounds described herein (such as salts, pro-drugs, metabolites, esters, ethers, hydrates, polymorphs, solvates, complexes, and adducts) which, upon administration to a subject, are capable of providing (directly or indirectly) the compounds described herein. As used herein, the term “pharmaceutically acceptable salt” refers to those salts, which retain the biological effectiveness and properties of the parent compound. Unless otherwise indicated, a pharmaceutically acceptable salt includes salts of acidic or basic groups, which may be present in the compounds of the formulae disclosed herein. The present disclosure also provides certain processes, as examples, for the preparation of the above pharmaceutically acceptable salts, their derivatives, their analogs, their tautomeric forms, their stereoisomers, their polymorphs, and pharmaceutical compositions containing them.

Certain embodiments of the present disclosure relate to pharmaceutically acceptable salts formed by the compounds described herein, or their derivatives, their analogs, their tautomeric forms, their stereoisomers, their polymorphs and pharmaceutically acceptable compositions containing them. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenyl sulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

Combinations containing the label-chelator-nucleoside analog conjugates combine imaging with the therapeutic intervention and can image in real time the uptake and activity of N4 conjugated nucleoside analog, which is essential to select the individual patient with the targeted dysfunctional pathway (right disease) and to assess optimal dosage (right dose). This approach allows for visually seeing the composition located at the tissue site and determining the actual dose of uptake to that site for that patient. This platform allows one to evaluate: (a) if dosing is the cause of the adverse event; (b) if bioavailability is the cause or (c) if there is a limited uptake and/or bio-distribution. In keeping with these parameters, the embodiments serve to dissect effects that are patient dependent, particularly if one of these effects are genetic, epigenetic, or exhibit allelic variations associated to the individual's ECS.

The effective amount of a compound is determined based on several factors, such as age and weight of the patient, severity of the disease, other co-existing factors. The effective amount of a compound includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition.

The following descriptions of methods, compositions, and results obtained using them are provided merely as illustrative examples. Descriptions of the methods are not intended to require or imply that the steps of the various embodiments must be performed in the order presented. The steps in the foregoing embodiments may be performed in any order. Words such as “then” are not intended to limit the order of the steps; these words are simply used to guide the reader through the description of the methods. Many of the operations may be performed in parallel or concurrently. In addition, the order of the operations may be re-arranged. Various modifications to these embodiments will be readily apparent based on the description provided here, and the generic principles defined here may be applied to other embodiments without departing from the scope of the disclosure.

Further modifications and alternative embodiments of various aspects of the compositions and methods disclosed here will be apparent in view of this description. Accordingly, this description is to be construed as illustrative only and is for the purpose of teaching those skilled in the art the general manner of carrying out the embodiments. It is to be understood that the forms of the embodiments shown and described here are to be taken as examples of embodiments. Elements and materials may be substituted for those illustrated and described here, parts and processes may be reversed or omitted, and certain features of the embodiments may be utilized independently, all as would be apparent after having the benefit of this description of the embodiments. Changes may be made in the elements described here without departing from the scope of the embodiments as described in the following claims.

### EXAMPLES

### Example 1— Synthesis of the Compound According to Formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide

The guanine nucleoside analog compound according to Formula I,

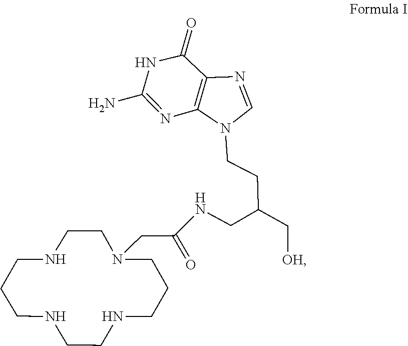 [SMILES: Nc1nc2c(ncn2CCC(CO)CNC(=O)CN2CCCNCCNCCCNCC2)c(=O)[nH]1]

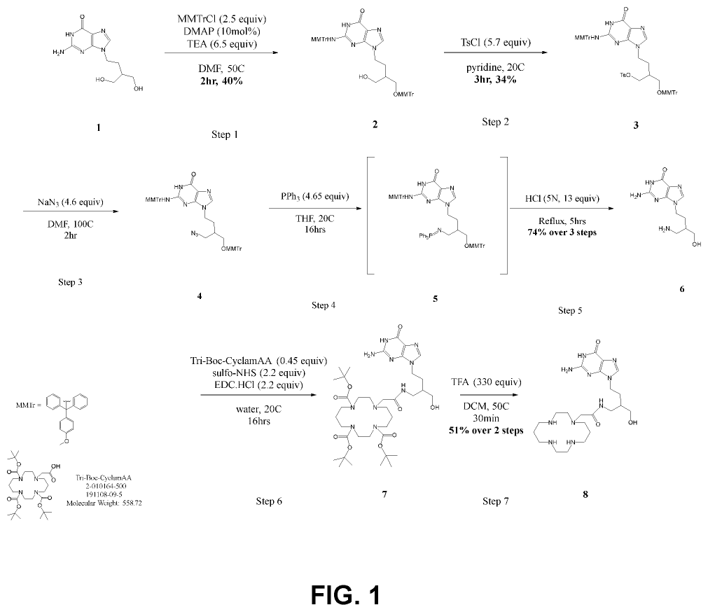

may be synthesized in several ways. FIG. 1 depicts an example process for the synthesis of the compound according to Formula I, N-(4-(2-amino-6-oxo-1,6,-dihydro-9H-purin-9-yl)-2-(hydroxymethyl)butyl)-2-(1,4,8,11-tetraazacyclotetradecan-1-yl)acetamide. As shown in FIG. 1, Compound 1 (penciclovir, 50.0 g, 1 Eq, 197 mmol) may be charged in a 1 L flask with a mechanical stirrer, thermocouple, and nitrogen inlet, followed by the addition of DMSO (300 mL, 60093) (dried over 4 A MS) and followed by the addition of triethylamine (44.0 g, 60.5 mL, 2.2 Eq, 434 mmol).

The mixture may be stirred for 10 min to give a white suspension. The stirring may then be increased to vigorous (400-500 RPM). MMTrCl (122 g, 2.0 Eq, 395 mmol) may then be added as a solid while maintaining temperature at 20-25° C. over 20 minutes. An ice bath is then used to periodically lower the reaction temperature. Following the addition, the reaction mixture is a thick brown-black solution. After 4 hrs, the reaction mixture is poured into a mixture of 1.5 L DCM and 1 L water. The reaction mixture is then stirred for 5-10 minutes and let settle for 1 hr before separating the layers. The organic layer is diluted with 1 L water, stirred for 5-10 minutes and let settle for 1 hr. After 1 hr, the mixture is filtered and the solids are discarded. The layers are separated and the organic layer is diluted with 1 L water, stirred for 5-10 minutes and let settle for 1 hr. The layers are separated and after 18 hr aging, the organic layer is filtered and the solids discarded. The organic layer is then dried over sodium sulfate (75 g) and filtered and evaporated filtrate on rotavap (50 mBar, 35° C.). Dried briefly under direct vacuum to give 150 g crude solids. Purified by flash chromatography on a 1.5 kg Biotage SNAP Ultra (25 uM) cartridge. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

Compound 2 (450 mg, 1 Eq, 564 μmol) was dissolved in Pyridine (7.5 mL) in a reaction vial with stir bar, thermocouple. Then p-toluenesulfonyl chloride (613 mg, 5.7 Eq, 3.21 mmol) was added over 10 min (9:40 AM-9:50 AM). Color darkens somewhat, very mild exotherm. Temperature remains between 20-22° C. After 3.5 hrs, diluted reaction mixture with EtOAc (20 mL) and water (10 mL). Wash organic with a further 2×10 mL water. Dried organic layer over Na2SO4 (500 mg-1 g), evaporated to dryness. Azeotroped 2×10 mL toluene. Then azeodry 1×10 mL MeCN to yield a yellow solid. Silica chromatography (14×) using 10 g cartridge, Biotage SNAP ultra. Reaction/column monitoring at 254 nm with lambda all detection. solvent. Dissolve in 1 mL EtOAc, liquid loading. Rinse 2 mL 65% EtOAc/heptane. MPA:hept. MPB:EtOAc. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

In a 1 L RBF with stir bar, thermocouple, nitrogen inlet dissolved Compound 3 (53.7 g, 1 Eq) in anhydrous DMF (537 mL, stored over 4 A MS) then added sodium azide (5.13 g, 1.4 Eq). Heated to 50° C. After heating for 24 hrs, cooled reaction to room temperature. partitioned mixture between EtOAc (1.5 L) and water (1.5 L). Let settle 1 hr, then split layers. Wash organic 2×1.5 L water further, allowing mixture to settle for 1 hr each time and discarding the rag layer. Dried organic layer over sodium sulfate (57 g). Evaporated on rotavap (35° C., 50 mBar) and dried briefly under direct vacuum to give Compound 4 as a white semisolid, 38.9 g, 75% yield. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

In a 50 mL RBF with stir bar, condenser, thermocouple, heating mantle, charge Compound 4 (1.00 g, 1 Eq) then THF (15 mL) and water (1.5 mL). Triphenylphosphine (344 mg, 1.2 Eq) was added, and the mixture was heated to 65° C. After 4 hrs, cool reaction to 25° C., add hydrochloric acid (216 mg, 0.18 mL, 2 Eq). The mixture was heated to 65° C. After 3 hrs, cool to room temperature and filter thru 0.2 uM frit. Separated colorless lower layer, transferred to RBF and evaporated to white residue. Dried in vacuum oven (20° C., −29 inHg) overnight to give Compound 6 as a white solid (351 mg). A qNMR experiment indicates that the material is 62% potent, with the remainder of mass being water (78% adjusted yield). The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

In a 100 mL RBF, dilute Compound 6 aqueous solution (35.91 g, 29.8 wt %) with 24 g water. Basify to pH 8.1 with 4 M NaOH. After 1.5 hrs, add 2 g of celite and filter suspension. Dry solids overnight at ambient temperature (−29 inHg) to give Compound 6 as a white solid. Solids were dissolved in 550 mL 20% DMSO/MeOH and filtered. Filtrate was evaporated on a rotavap (40° C., 50 mBar) and then under direct vacuum to give Compound 6 solution in DMSO (41.61 g, 12.6 wt %). Compound 6 DMSO solution (41.61 g, 12.6 wt %) was further diluted with anhydrous DMSO (73 mL) and then anhydrous DMF (212 mL). Added stir bar, nitrogen inlet, thermocouple. Added TriBocCyclamAA (12.9 g, 1.1 Eq) then DMAP (5.13 g, 2.0 Eq) and stirred until mostly dissolved. Then, added EDC.HCl (8.1 g, 2.0 Eq) in a single portion at 20° C. After 24 hrs, the reaction mixture was diluted with DCM (815 mL), 160 mL water, and 650 mL sat. sodium sulfate. The pH of the aqueous layer was adjusted from 8 to 4 using 6 M HCl (−4.5 mL). The biphase was allowed to settle for 1 hr, then the layers were separated. The organic was washed 4× further with 160 mL water, 650 mL sat. sodium sulfate, maintaining the aqueous pH between 4-5 using 6 M HCl. The organic layer was dried over sodium sulfate (27 g) and filtered. The sodium sulfate cake was rinsed with 150 mL DCM and the filtrate evaporated on a rotavap (50 mBar, 40° C.) then under direct vacuum (−29 inHg) to give 31.34 g pale yellow oil (49.1 wt %, 15.4 g intermediate 6, 92% yield). The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

As further shown in FIG. 1, Compound 7 is dissolved (13.2 g, 1 Eq) in DCM (190 mL) and MeCN (20 mL) in 1 L flask with stir bar. Add triethylsilane (19.9 mL, 7.5 Eq) then cool to 0° C. Add trifluoroacetic acid (51.3 mL, 40 Eq) maintaining temperature <10° C. Following addition warm to room temperature. After 23 hr, charge additional trifluoroacetic acid (12.5 mL, 10 Eq). After 24 hrs, dilute mixture with 40 mL water, stir for 1.5 hr, then let settle for 15 min. Collect faint purple, hazy aqueous lower layer into 1 L RBF. Extract organic additional portion 40 mL water, combine colorless upper aqueous layer with previous aqueous extract. Adjust pH to 8.4 with 4 M NaOH, maintaining temperature <35° C. Strip on rotavap (40° C., 50 mBar) then freeze dry overnight to give 180 g crude as an aqueous solution. The resulting compound was analyzed by proton nuclear magnetic resonance (¹H NMR), carbon-13 nuclear magnetic resonance (¹³C NMR), and high-resolution mass spectrometry (HRMS) by electrospray ionization (ESI).

### Example 2— Synthesis of the Compound According to Formula II, N-(9-(4-amino-3-(hydroxymethyl)butyl)-6-oxo-6,9-dihydro-1H-purin-2-yl)-2-(1,4,8,11-tetraazacyclotetradecan-1-yl)acetamide

Synthesis affords the constitutional isomer nucleoside analog compound according to Formula II,

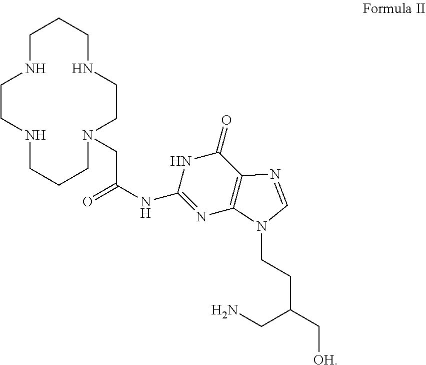 [SMILES: NCC(CO)CCn1cnc2c(=O)[nH]c(NC(=O)CN3CCCNCCNCCCNCC3)nc21]

### Example 3—Synthesis of the Compound According to Formula III, N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide

Synthesis affords the dicyclam product nucleoside analog compound according to Formula III,

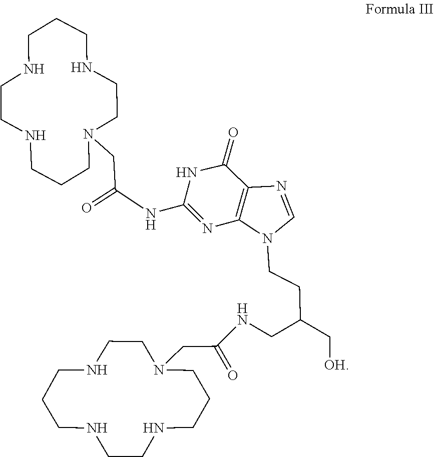 [SMILES: O=C(CN1CCCNCCNCCCNCC1)NCC(CO)CCn1cnc2c(=O)[nH]c(NC(=O)CN3CCCNCCNCCCNCC3)nc21]

### Example 4—Synthesis of the Compound According to Formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide]

The nucleoside analog compound according to Formula IV,

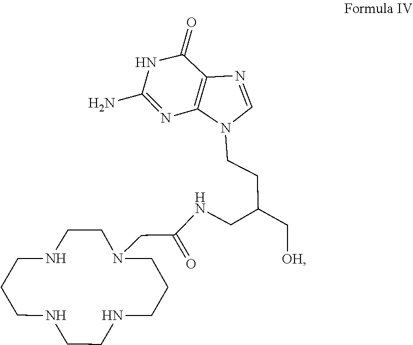 [SMILES: Nc1nc2c(ncn2CCC(CO)CNC(=O)CN2CCCNCCNCCCNCC2)c(=O)[nH]1]

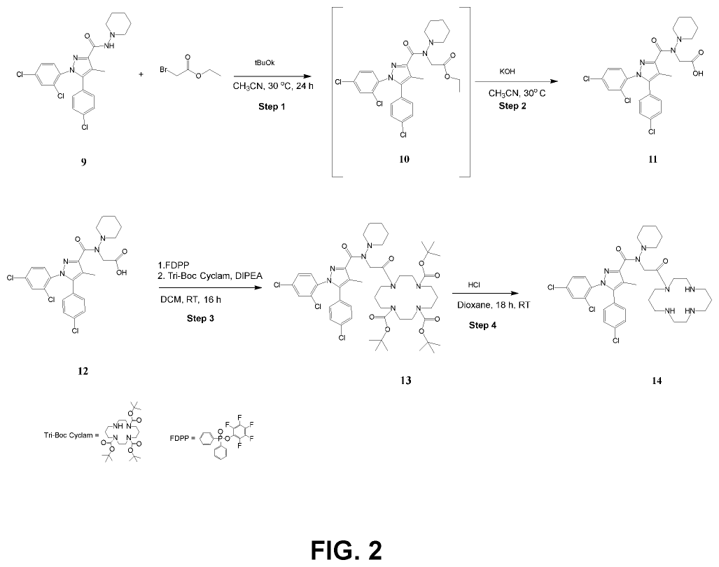

may be synthesized in several ways. FIG. 2 depicts an example process for the synthesis of the compound according to Formula IV, 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide].

### Statements of the Disclosure Include

Statement 1: A theranostic radiopharmaceutical composition comprising: a conjugate of a nucleoside analog, a chelator, and a label.

Statement 2: The theranostic radiopharmaceutical composition according to Statement 1, wherein the nucleoside analog is a guanine analog.

Statement 3: The theranostic radiopharmaceutical composition according to Statement 1, wherein the nucleoside analog is a cell replication check point ligand.

Statement 4: The theranostic radiopharmaceutical composition according to Statement 1, wherein the nucleoside analog is a synthetic analog.

Statement 5: The theranostic radiopharmaceutical composition according to Statement 1, wherein the nucleoside analog is a natural analog.

Statement 6: The theranostic radiopharmaceutical composition according to Statement 1, wherein the nucleoside analog is guanine.

Statement 7: The theranostic radiopharmaceutical composition according to Statement 1, wherein the nucleoside analog is selected from the group consisting of adenine, adenosine, deoxyadenosine, guanine, guanosine, deoxyguanosine, thymine, 5-methyluridine, thymidine, uracile, uridine, deoxyuridine, cytosine, cytidine, deoxycytidine, and any combination thereof.

Statement 8: The theranostic radiopharmaceutical composition according to Statement 1, wherein the nucleoside analog is arabinosyl nucleoside.

Statement 9: The theranostic radiopharmaceutical composition according to any one of Statements 1-8, wherein the chelator is an aminated chelator.

Statement 10: The theranostic radiopharmaceutical composition according to any one of Statements 1-8, wherein the chelator is an acid chelator.

Statement 11: The theranostic radiopharmaceutical composition according to any one of Statements 1-8, wherein the chelator is cyclam.

Statement 12: The theranostic radiopharmaceutical composition according to any one of Statements 1-8, wherein the chelator is a N4 chelator or ligand.

Statement 13: The theranostic radiopharmaceutical composition according to any one of Statements 1-8, wherein the chelator is 6-carboxy-1,4,8,11-tetraazaundecane.

Statement 14: The theranostic radiopharmaceutical composition according to any one of Statements 1-8, wherein the chelator is 1,4,8,11-tetraazabicyclohexadecane.

Statement 15: The theranostic radiopharmaceutical composition according to any one of Statements 1-14, wherein the label is a radiotracer label.

Statement 16: The theranostic radiopharmaceutical composition according to any one of Statements 1-15, wherein the label is a radionuclide.

Statement 17: The theranostic radiopharmaceutical composition according to any one of Statements 1-16, wherein the label is a radionuclide metal ion.

Statement 18: The theranostic radiopharmaceutical composition according to any one of Statements 1-16, wherein the label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

Statement 19: The theranostic radiopharmaceutical composition according to any one of Statements 1-15, wherein the label is configured to facilitate contrast-enhanced imaging when administered to a mammalian subject in conjunction with diagnostic imaging.

Statement 20: The theranostic radiopharmaceutical composition according to any one of Statements 1-19, wherein the conjugate comprises N4-guanine (N4amG).

Statement 21: The theranostic radiopharmaceutical composition according to any one of Statements 1-19, wherein the conjugate comprises cyclam-am-guanine.

Statement 22: The theranostic radiopharmaceutical composition according to any one of Statements 1-19, wherein the conjugate comprises N-(4-(2-amino-6-oxo-1,6,-dihydro-9H-purin-9-yl)-2-(hydroxymethyl)butyl)-2-(1,4,8,11-tetraazacyclotetradecan-1-yl)acetamide.

Statement 23: The theranostic radiopharmaceutical composition according to any one of Statements 1-19, wherein the conjugate comprises a conjugate compound having a structure according to Formula I:

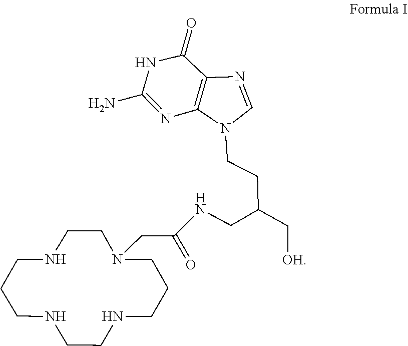 [SMILES: Nc1nc2c(ncn2CCC(CO)CNC(=O)CN2CCCNCCNCCCNCC2)c(=O)[nH]1]

Statement 24: The theranostic radiopharmaceutical composition according to any one of Statements 1-19, wherein the conjugate comprises N-(9-(4-amino-3-(hydroxymethyl)butyl)-6-oxo-6,9-dihydro-1H-purin-2-yl)-2-(1,4,8,11-tetraazacyclotetradecan-1-yl)acetamide.

Statement 25: The theranostic radiopharmaceutical composition according to any one of Statements 1-19, wherein the conjugate comprises a conjugate compound having a structure according to Formula II:

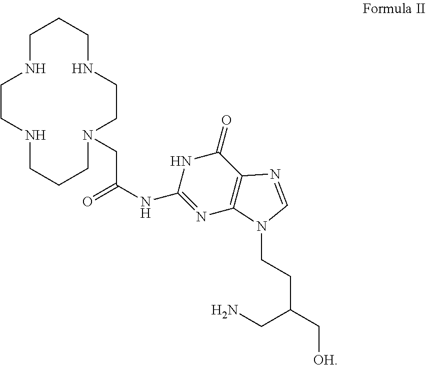 [SMILES: NCC(CO)CCn1cnc2c(=O)[nH]c(NC(=O)CN3CCCNCCNCCCNCC3)nc21]

Statement 26: The theranostic radiopharmaceutical composition according to any one of Statements 1-19, wherein the conjugate comprises N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide.

Statement 27: The theranostic radiopharmaceutical composition according to any one of Statements 1-19, wherein the conjugate comprises a conjugate compound having a structure according to Formula III:

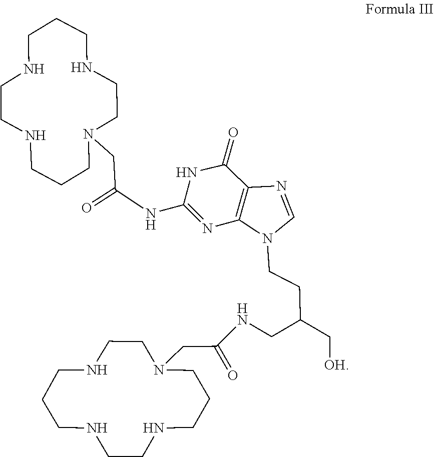 [SMILES: O=C(CN1CCCNCCNCCCNCC1)NCC(CO)CCn1cnc2c(=O)[nH]c(NC(=O)CN3CCCNCCNCCCNCC3)nc21]

Statement 28: The theranostic radiopharmaceutical composition according to any one of Statements 1-19, wherein the conjugate comprises 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide].

Statement 29: The theranostic radiopharmaceutical composition according to any one of Statements 1-19, wherein the conjugate comprises a conjugate compound having a structure according to Formula IV:

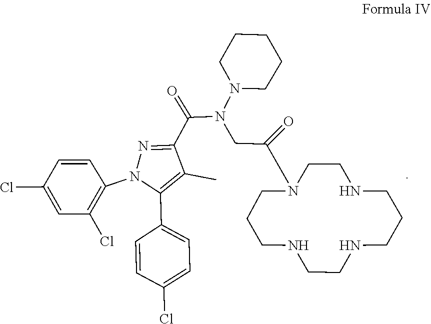 [SMILES: Cc1c(C(=O)N(CC(=O)N2CCCNCCNCCCNCC2)N2CCCCC2)nn(-c2ccc(Cl)cc2Cl)c1-c1ccc(Cl)cc1]

Statement 30: A method of diagnosing and treating an infectious disease in a subject in need thereof using a theranostic radiopharmaceutical, the method comprising: administering to the subject a pharmaceutically effective amount of a theranostic radiopharmaceutical composition according to any one of Statements 1-29; and performing an imaging technique on the subject or a portion thereof, wherein the imaging technique is capable of detecting one or more signals from the theranostic radiopharmaceutical composition.

Statement 31: A method of monitoring and treating an infectious disease in a subject in need thereof using a theranostic radiopharmaceutical, the method comprising: administering to the subject a pharmaceutically effective amount of a theranostic radiopharmaceutical composition according to any one of Statements 1-29; and performing an imaging technique on the subject or a portion thereof, wherein the imaging technique is capable of detecting one or more signals from the theranostic radiopharmaceutical composition.

Statement 32: A method of imaging and treating a plurality of cells in a subject wherein the plurality of cells are infected with an infectious disease pathogen, the method comprising: administering to the subject a pharmaceutically effective amount of a theranostic radiopharmaceutical composition according to any one of Statements 1-29 in a manner such that the plurality of cells effectively receive the theranostic radiopharmaceutical composition; and performing an imaging technique on at least a portion of the subject containing the plurality of cells, wherein the imaging technique is capable of detecting one or more signals from the theranostic radiopharmaceutical composition.

Statement 33: The method according to any one of Statements 30-32, further comprising: making at least one treatment decision based on the results of the imaging technique performed on the subject.

Statement 34: The method according to any one of Statements 30-33, wherein the imaging technique is selected from the group consisting of positron emission tomography (PET), computed tomography (CT), single photon emission computed tomography (SPECT), magnetic resonance imaging (MM), near-infrared (NIR), optical imaging, optoacoustic imaging, ultrasound, and any combination thereof.

Statement 35: The method according to any one of Statements 30-34, wherein the infectious disease is a viral infection.

Statement 36: The method according to any one of Statements 30-34, wherein the infectious disease is a respiratory viral infection selected from the group consisting of human influenza, the common cold, Middle East respiratory syndrome (MERS), severe acute respiratory syndrome coronavirus (SARS), and COVID-19.

Statement 37: The method according to any one of Statements 30-34, wherein the infectious disease is caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus OC43 (HCoV-OC43), human coronavirus HKU1 (HCoV HKU1), and human coronavirus 229E (HCoV-229E).

Statement 38: The method according to any one of Statements 30-37, wherein the label is a radiotherapeutic label capable of delivering radiation to a tissue in the subject targeted by the conjugate.

Statement 39: The method according to Statement 38, wherein the label is a radiotherapeutic label capable of delivering radiation to an infected tissue in the subject and the advantageous therapeutic effect is reduced inflammation, increased viral particle inactivation, reduced viral replication, and/or reduced viral load in the tissue.

Statement 40: The method according to Statement 38 or Statement 39, wherein the radiotherapeutic label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

What is claimed is:

1. A theranostic radiopharmaceutical composition comprising:
a conjugate of a nucleoside analog, a chelator, and a label;
wherein the label is a radiotherapeutic label capable of delivering radiation to a tissue in the subject targeted by the conjugate, the label further configured to facilitate contrast-enhanced imaging when administered to a mammalian subject in conjunction with diagnostic imaging.

2. The theranostic radiopharmaceutical composition according to claim 1, wherein the label is a radiotherapeutic label capable of delivering radiation to an infected tissue in the subject and producing at least one advantageous therapeutic effect selected from the group consisting of reduced inflammation, increased viral particle inactivation, reduced viral replication, and/or reduced viral load in the tissue.

3. The theranostic radiopharmaceutical composition according to claim 1, wherein the label is selected from the group consisting of Technetium-99, Gallium-68, Copper-60, Copper-64, Indium-111, Holmium-166, Rhenium-186, Rhenium-188, Yttrium-90, Lutetium-177, Radium-223, Actinium-225, and any combination thereof.

4. The theranostic radiopharmaceutical composition according to claim 3, wherein the nucleoside analog is selected from the group consisting of adenine, adenosine, deoxyadenosine, guanine, guanosine, deoxyguanosine, thymine, 5-methyluridine, thymidine, uracile, uridine, deoxyuridine, cytosine, cytidine, deoxycytidine, and any combination thereof.

5. The theranostic radiopharmaceutical composition according to claim 4, wherein the chelator is selected from the group consisting of an aminated chelator, an acid chelator, a cyclam, a N4 chelator or ligand, 6-carboxy-1,4,8,11-tetraazaundecane, 1,4,8,11-tetraazabicyclohexadecane, and any combination thereof.

6. The theranostic radiopharmaceutical composition according to claim 1, wherein the conjugate comprises N4-guanine (N4amG).

7. The theranostic radiopharmaceutical composition according to claim 1, wherein the conjugate comprises cyclam-am-guanine.

8. The theranostic radiopharmaceutical composition according to claim 1, wherein the conjugate comprises N-(4-(2-amino-6-oxo-1,6,-dihydro-9H-purin-9-yl)-2-(hydroxymethyl)butyl)-2-(1,4,8,11-tetraazacyclotetradecan-1-yl)acetamide.

9. The theranostic radiopharmaceutical composition according to claim 1, wherein the conjugate comprises a conjugate compound having a structure according to Formula I:

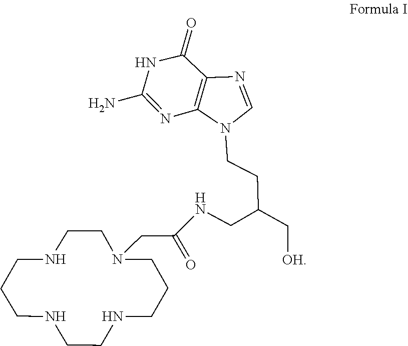 [SMILES: Nc1nc2c(ncn2CCC(CO)CNC(=O)CN2CCCNCCNCCCNCC2)c(=O)[nH]1]

10. The theranostic radiopharmaceutical composition according to claim 1, wherein the conjugate comprises N-(9-(4-amino-3-(hydroxymethyl)butyl)-6-oxo-6,9-dihydro-1H-purin-2-yl)-2-(1,4,8,11-tetraazacyclotetradecan-1-yl)acetamide.

11. The theranostic radiopharmaceutical composition according to claim 1, wherein the conjugate comprises a conjugate compound having a structure according to Formula II:

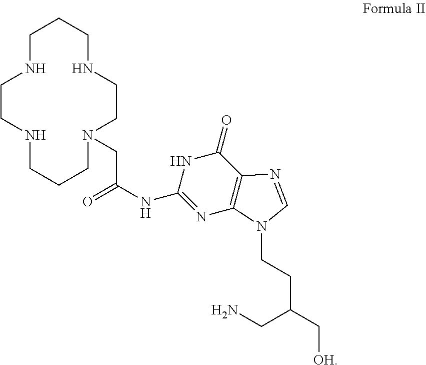 [SMILES: NCC(CO)CCn1cnc2c(=O)[nH]c(NC(=O)CN3CCCNCCNCCCNCC3)nc21]

12. The theranostic radiopharmaceutical composition according to claim 1, wherein the conjugate comprises N-(9-(4-(2-(1,4,8,11-tetraazacyclotetradecan-1-yl)acetamido-3-(hydroxymethyl)butyl)-6-oxo-6,9-dihydro-1H-purin-2-yl)-2-(1,4,8,11-tetraazacyclotetradecan-1-yl)acetamide.

13. The theranostic radiopharmaceutical composition according to claim 1, wherein the conjugate comprises a conjugate compound having a structure according to Formula III:

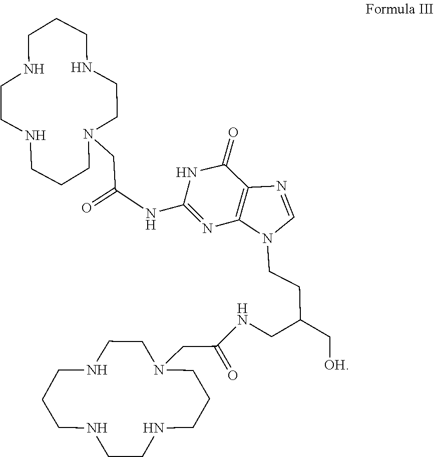 [SMILES: O=C(CN1CCCNCCNCCCNCC1)NCC(CO)CCn1cnc2c(=O)[nH]c(NC(=O)CN3CCCNCCNCCCNCC3)nc21]

14. The theranostic radiopharmaceutical composition according to any one of claim 1, wherein the conjugate comprises 1,4,8,11-tetraazacyclotetradecane-1′-acetyl-[N-(Piperidin-1-yl)-5-(4-chlorophenyl)-1-(2,4-dichlorophenyl)-4-methyl-1H-pyrazole-3-carboxamide].

15. The theranostic radiopharmaceutical composition according to claim 1, wherein the conjugate comprises a conjugate compound having a structure according to Formula IV:

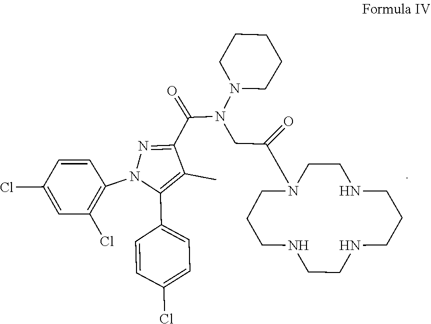 [SMILES: Cc1c(C(=O)N(CC(=O)N2CCCNCCNCCCNCC2)N2CCCCC2)nn(-c2ccc(Cl)cc2Cl)c1-c1ccc(Cl)cc1]

16. A method of contemporaneously diagnosing, treating, and monitoring an infectious disease in a subject in need thereof using a theranostic radiopharmaceutical, the method comprising:
administering to the subject a pharmaceutically effective amount of a theranostic radiopharmaceutical composition according to claim 5; and
performing an imaging technique on the subject or a portion thereof, wherein the imaging technique is capable of detecting one or more signals from the theranostic radiopharmaceutical composition.

17. The method according to claim 16, further comprising:
making at least one treatment decision based on the results of the imaging technique performed on the subject.

18. The method according to claim 16, wherein the imaging technique is selected from the group consisting of positron emission tomography (PET), computed tomography (CT), single photon emission computed tomography (SPECT), magnetic resonance imaging (MRI), near-infrared (NIR), optical imaging, optoacoustic imaging, ultrasound, and any combination thereof.

19. The method according to claim 16, wherein the infectious disease is a respiratory viral infection selected from the group consisting of human influenza, the common cold, Middle East respiratory syndrome (MERS), severe acute respiratory syndrome coronavirus (SARS), and COVID-19.

20. The method according to claim 19, wherein the infectious disease is caused by infection by a virus selected from the group consisting of severe acute respiratory syndrome coronavirus (SARS-CoV), severe acute respiratory syndrome coronavirus 2 (SARS-CoV-2), Middle East respiratory syndrome-related coronavirus (MERS-CoV), human coronavirus NL63 (HCoV NL63), human coronavirus OC43 (HCoV-OC43), human coronavirus HKU1 (HCoV HKU1), and human coronavirus 229E (HCoV-229E).
